(19) United States
(12) Patent Application Publication
(10) Pub. No.: US 2008/0314734 A1
(43) Pub. Date: Dec. 25, 2008

(54) Carbonaceous solid fuel gasifier utilizing dielectric barrier non-thermal plasma

(75) Inventors: Yong Ho Kim, Los Alamos, NM (US); Louis A. Rosocha, Los Alamos, NM (US); Graydon K. Anderson, Santa Fe, NM (US); Hans-Joachim Ziock, Los Alamos, NM (US); Sara Abbate, Chatillon (FR); Rodrigo Sanchez-Gonzalez, Queretaro (MX)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: The Regents of the University of California

(21) Appl. No.: 11/894,633

(22) Filed: Aug. 8, 2007

Related U.S. Application Data
(60) Provisional application No. 60/936,961, filed on Jun. 21, 2007.

(51) Int. Cl. C01B 3/02; C07C 2/00; B01J 19/08

(52) U.S. Cl. 204/170; 204/164; 422/186.04

(57) ABSTRACT

A system for producing a fuel gas from a carbon-containing material is provided that includes a non-thermal plasma generator, an electric power source, a process stream inlet, and a product stream outlet. The non-thermal plasma generator includes a high voltage electrode separated from a grounded electrode by a modification passage. Moreover, a dielectric layer exists between the high voltage electrode and the grounded electrode. The electric power source is energizable to create non-thermal electrical microdischarges within the modification passage. As the process gas flows through the system, the carbon-containing material is converted to fuel gas.

### RELATED APPLICATIONS

This application claims the benefit of U.S. Provisional Application No. 60/936,961, filed Jun. 21, 2007.

### STATEMENT OF FEDERAL RIGHTS

The United States government has rights in this invention pursuant to Contract No. DE-AC52-06NA25396 between the United States Department of Energy and Los Alamos National Security, LLC for the operation of Los Alamos National Laboratory.

### FIELD OF INVENTION

The present invention pertains generally to a system and process for producing a fuel gas from a carbon-containing material, and more particularly to non-thermal plasma reactors.

### BACKGROUND

Gasification of carbonaceous solid fuels, such as coal and biomass, has become of increasing interest and importance because of rapidly rising petroleum prices, dwindling domestic petroleum and natural gas resources, and the increased dependency by the United States on foreign petroleum imports. Gasification of coal and biomass has been widely practiced for over 100 years, and there are many varieties and types of gasifiers and routes to gasification.

A conventional gasification process, however, typically involves a hostile environment that includes high temperatures, possibly high pressures, abrasion, and poisoning of catalysts by sulfur or other contaminants in the solid fuel. Moreover, such severe operating conditions rapidly degrade the costly conventional catalysts required for practical implementation of gasification. Thus, there remains a need for robust technologies that can make gasification efficient, fast, inexpensive, and resilient enough to run problem-free on solid “dirty” fuels.

### SUMMARY OF INVENTION

The present invention overcomes many of these drawbacks and enables the end user to effectively produce a fuel gas from a carbon-containing material in a timely and economical fashion.

By way of example, and not of limitation, the present invention is a system that employs electrical discharges/non-thermal plasmas in a gaseous medium to convert carbon-containing material into a fuel gas. In non-thermal plasmas, the electrons are “hot,” while the ions and neutral species are “cold” which results in little waste enthalpy being deposited in a process stream. This is in contrast to thermal plasmas where the electron, ion, and neutral-species' energies are “hot” and considerable waste heat is deposited in the process stream. The present invention utilizes the non-thermal plasma (and its associated energetic electrons, highly reactive free radicals, and minimal waste enthalpy) by having it serve as a catalyst to promote the gasification reaction. This can provide advantages in terms of lowering the temperature and pressure required to overcome the activation energy barrier, and, thereby, improving the conversion of carbon-containing material to a usable fuel gas.

In the present invention, the non-thermal plasma is generated by microdischarges generated by a dielectric barrier discharge/silent discharge plasma. A dielectric barrier discharge is a type of electrical discharge that occurs in an open space between two insulated electrodes connected to a source of high voltage alternating current. Such discharges are commonly created in a dielectric barrier electrode arrangement in which one or both metal electrodes are covered with materials with a high dielectric constant. A thin gas layer separates the electrodes; however, the dielectric material may also be placed between the electrodes to separate two gas layers.

In the present invention, the non-thermal plasma generator may have a variety of shapes including, but not limited to, planar and cylindrical. Examples of dielectric barrier configurations include, but are not limited to, plates, half-box shapes, C-shapes, and tubes.

In the present invention, the fuel gas may comprise a variety of gases including, but not limited to, hydrogen, carbon monoxide, synthesis gas, methane, and other hydrocarbons. The carbon-containing material may have a variety of forms including, but not limited to, carbonaceous solid fuels such as biomass, carbon particles, coal, and coke. In one aspect of the present invention the carbon-containing material is crushed and sieved to a size smaller than about 1 millimeter (“mm”). In another aspect of the present invention the carbon-containing material is pulverized to sizes from around 10 to 100 micrometers (“μm”).

In one aspect of the present invention, a system for producing a fuel gas from a carbon-containing material comprises a non-thermal plasma generator, an electric power source for providing an electrical charge to the non-thermal plasma generator, a process stream inlet configured to inject a process stream into the non-thermal plasma generator, and a product stream outlet configured to remove a product stream from the non-thermal plasma generator. The process stream comprises hydrogen, steam, or carbon dioxide, and the product stream comprises the fuel gas. The non-thermal plasma generator comprises a high voltage electrode, a grounded electrode spaced apart from the high voltage electrode, a first dielectric layer between the high voltage electrode and the grounded electrode, and a modification passage between the high voltage electrode and the grounded electrode. The process stream inlet is configured to inject the process stream into the modification passage, and the high voltage electrode is connected to the electric power source and is characterized as energizable to create non-thermal electrical microdischarges between the high voltage electrode and the grounded electrode across the first dielectric layer. Moreover, the carbon-containing material is within the modification passage thereby creating a batch system with respect to the carbon-containing material. In the modification passage, the process stream reacts with the carbon-containing material to yield the product stream comprising the fuel gas.

In another aspect of the present invention, a system for producing a fuel gas from a carbon-containing material comprises a non-thermal plasma generator, an electric power source for providing an electrical charge to the non-thermal plasma generator, a process stream inlet configured to inject a process stream into the non-thermal plasma generator, and a product stream outlet configured to remove a product stream from the non-thermal plasma generator. The process stream comprises hydrogen, steam, or carbon dioxide, and the product stream comprises the fuel gas. The non-thermal plasma generator comprises a high voltage electrode, a grounded electrode spaced apart from the high voltage electrode, a first dielectric layer between the high voltage electrode and the grounded electrode, and a modification passage between the high voltage electrode and the grounded electrode. The process stream inlet is configured to inject the process stream into the modification passage, and the high voltage electrode is connected to the electric power source and is characterized as energizable to create non-thermal electrical microdischarges between the high voltage electrode and the grounded electrode across the first dielectric layer. Moreover, the process stream further comprises the carbon-containing material thereby creating a continuous system with respect to the carbon-containing material. In the modification passage, the process stream reacts to yield the product stream comprising the fuel gas.

In still another aspect of the present invention, a process for producing a fuel gas from a carbon-containing material comprises generating a non-thermal plasma, and contacting the carbon-containing material with the non-thermal plasma for a time and temperature sufficient to form the fuel gas.

The present invention can provide a more efficient system for producing a fuel gas from a carbon-containing material using a non-thermal plasma. The present invention allows the ability to construct a system for producing a fuel gas from a carbon-containing material in multiple geometries.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The present invention will be more fully understood by reference to the following drawings which are for illustrative purposes only.

FIG. 1 is an external side view of a first embodiment of the system for producing a fuel gas from a carbon-containing material.

FIG. 2 is a cutaway view of the first embodiment of the system for producing a fuel gas from a carbon-containing material taken along line 2-2 in FIG. 1.

FIG. 3 is a cutaway view of a second embodiment of the system for producing a fuel gas from a carbon-containing material.

FIG. 4 is a cutaway view of a third embodiment of the system for producing a fuel gas from a carbon-containing material.

FIG. 5 shows methane production as a function of oven temperature and plasma power.

### DETAILED DESCRIPTION

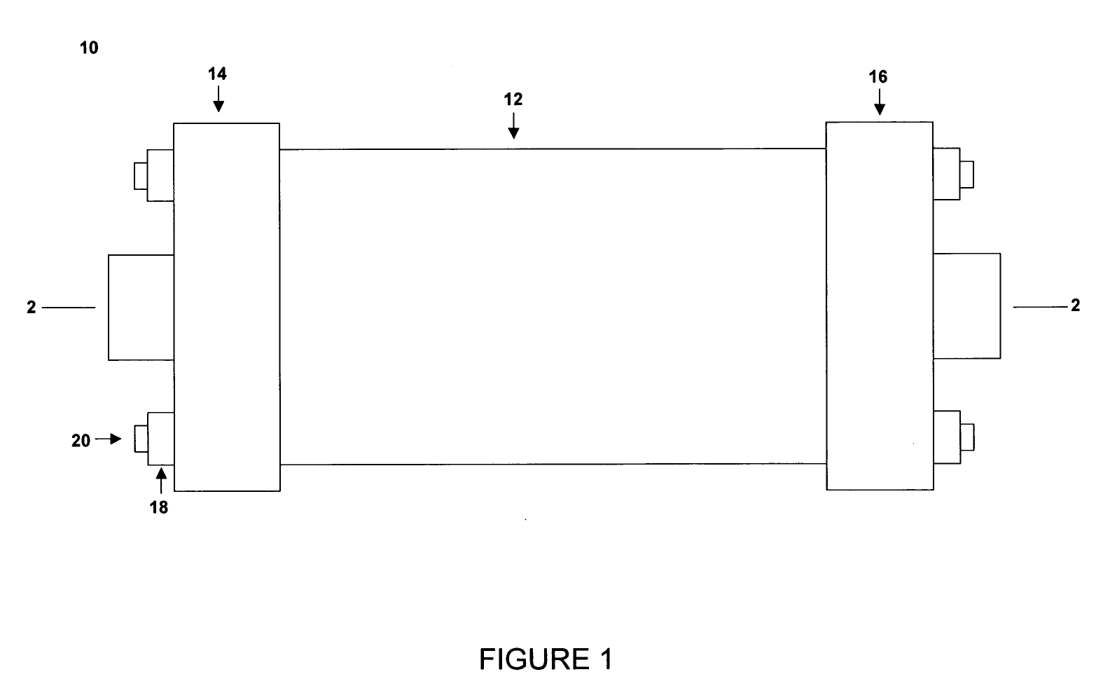

Referring more specifically to the drawings, for illustrative purposes the present invention is embodied in the systems shown in FIGS. 1 through 4. It will be appreciated that each apparatus of the invention may vary as to configuration and as to details of the parts, and that the process may vary as to the specific steps and sequence, without departing from the basic concepts as disclosed herein.

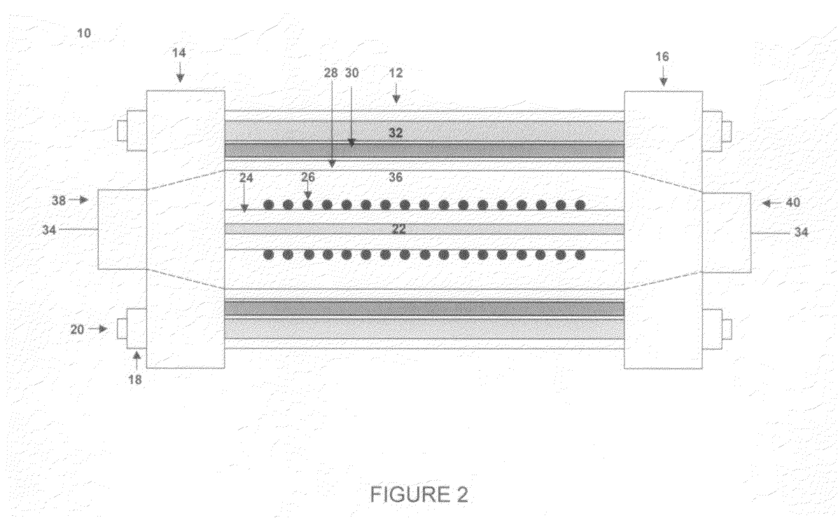

FIGS. 1 and 2 show an embodiment of the system for producing a fuel gas from a carbon-containing material, generally designated system 10. As shown in FIGS. 1 and 2, system 10 includes a generally cylindrical housing 12 disposed between a generally disk-shaped inlet end cap 14 and a generally disk-shaped outlet end cap 16. FIGS. 1 and 2 show that end caps 14, 16 can be removably engaged from housing 12 using plural nuts 18 and plural bolts 20, but it can be appreciated that any other fastening means well known in the art can be used.

FIG. 2 shows that system 10 includes an electrically conducting, generally cylindrical high voltage (“HV”) electrode 22 that is disposed within housing 12 between end caps 14, 16. In one embodiment, HV electrode 22 is connected to an alternating current (“AC”) source or a pulsed direct current (“DC”) source. Moreover, a generally cylindrical, dielectric tube 24 is disposed upon HV electrode 22 such that dielectric tube 24 closely surrounds HV electrode 22. Dielectric tube 24 is made from a dielectric material such as ceramic, glass, quartz, etc.

As shown in FIG. 2, a carbon-containing material 26 is disposed upon dielectric tube 24. A generally cylindrical, dielectric tube 28 surrounds dielectric tube 24. Similar to dielectric tube 24, dielectric tube 28 is made from a dielectric material such as ceramic, glass, quartz, etc. A generally cylindrical grounded electrode 30 is disposed upon dielectric tube 28 such that grounded electrode 30 closely surrounds the dielectric tube 28. FIG. 2 further shows that a heat exchanger 32 surrounds the grounded electrode 30. It is to be understood that the HV electrode 22, the dielectric tube 24, the dielectric tube 28, and the grounded electrode 30 are concentric to each other and are centered on a central axis 34.

FIG. 2 shows that a modification passage 36 is established between dielectric tube 24 and dielectric tube 28. Modification passage 36 is between one-half and several millimeters (e.g., ½ to 10 mm) wide. FIG. 2 further shows that system 10 includes a process stream inlet 38 established by inlet cap 14. Process stream inlet 38 leads to modification passage 36. The process stream comprises hydrogen, steam, or carbon dioxide. Also, a product stream outlet 40 is established by outlet end cap 16 and leads from modification passage 36. The product stream comprises a fuel gas. It is to be understood that the fuel gas produced depends upon the process stream composition.

It is to be understood that when HV electrode 22 is energized, non-thermal electrical microdischarges occur between HV electrode 22 and grounded electrode 30 across dielectric tube 24 and dielectric tube 28. The non-thermal electrical microdischarges generate a non-thermal plasma that occurs within modification passage 36. The non-thermal plasma directly converts some of the process stream into highly reactive chemical species, such as free radicals. These reactive species reduce the temperature and pressure required to overcome the activation energy barrier for production of a fuel gas from carbon-containing material 26. The result is the production of a product stream that exits modification passage 36 through product stream outlet 40.

It is well known to those skilled in the art that several types of electric discharge configurations can generate a non-thermal plasma. In this exemplary, non-limiting embodiment of the present invention, system 10 utilizes a dielectric-barrier discharge arrangement. The two electrodes (i.e., HV electrode 22 and grounded electrode 30) are separated by a relatively thin gas-containing space (i.e., modification passage 36). Both electrodes are covered by a dielectric material (i.e., dielectric tube 24 and dielectric tube 28, respectively). It can be appreciated that alternative dielectric-barrier discharge arrangements utilize planar or cylindrical arrangements with only one electrode (i.e., either HV electrode 22 or grounded electrode 30) covered with a dielectric material (i.e., dielectric tube 24 or dielectric tube 28, respectively). It can also be appreciated that the location of HV electrode 22 and grounded electrode 30 can be reversed.

A HV signal (e.g., alternating current with a frequency in a range from about 10 hertz (“Hz”) to about 20 kilohertz (“kHz”)) is applied to HV electrode 22 and grounded electrode 30 thereby creating electrical-discharge streamers (i.e., microdischarges) in modification passage 36. It is to be understood that the discharges generate the non-thermal plasma.

It is to be understood that system 10 is a batch system with respect to carbon-containing material 26. In one embodiment of system 10, the process stream is supplied to process stream inlet 38, and continuously flows through modification passage 36. After the reaction occurs, product stream exits through product stream outlet 40. Either circulating process stream or single-pass process stream may provide the continuous flow. In an alternative embodiment of system 10, the process stream is supplied to process stream inlet 38 and fills modification passage 36 until the desired system pressure is attained. Once the desired system pressure is attained, the process stream ceases to flow. After the reaction occurs, product stream exits through product stream outlet 40. In sum, system 10 is a batch system with respect to carbon-containing material 26, but may operate as either a continuous system (e.g., circulating or single-pass) or a batch system with respect to the process stream.

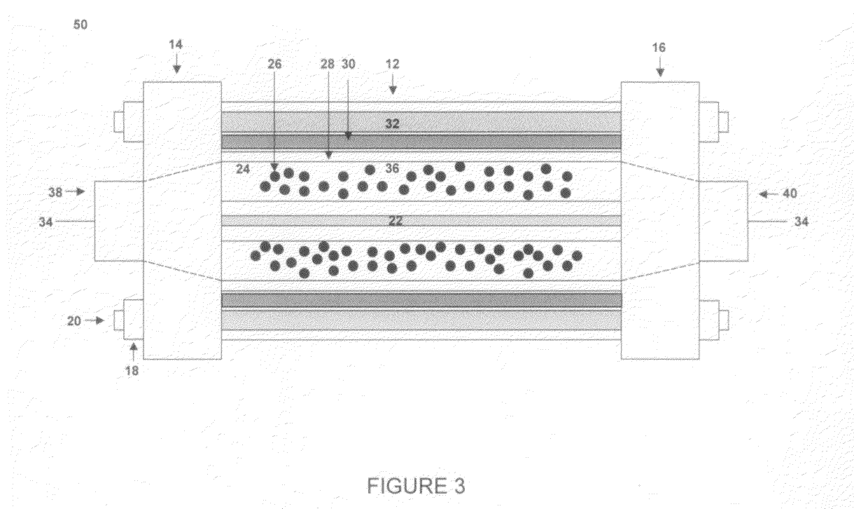

FIG. 3 shows another embodiment of the system for producing a fuel gas from a carbon-containing material, generally designated system 50. As shown in FIG. 3, system 50 is similar in every aspect to the system shown in FIGS. 1 and 2 except that modification passage 36 is packed with carbon-containing material 26. System 50 can be partially packed or fully packed with carbon-containing material 26. Carbon-containing material 26 ranges in diameter from about 10 μm to several millimeters. It is understood that system 50 can operate as either a continuous system (e.g., circulating or single-pass) or a batch system with respect to the process stream. It is also understood that system 50 can operate with an alternative dielectric-barrier discharge arrangement wherein only one electrode (i.e., either HV electrode 22 or grounded electrode 30) is covered with a dielectric material (i.e., dielectric tube 24 or dielectric tube 28, respectively).

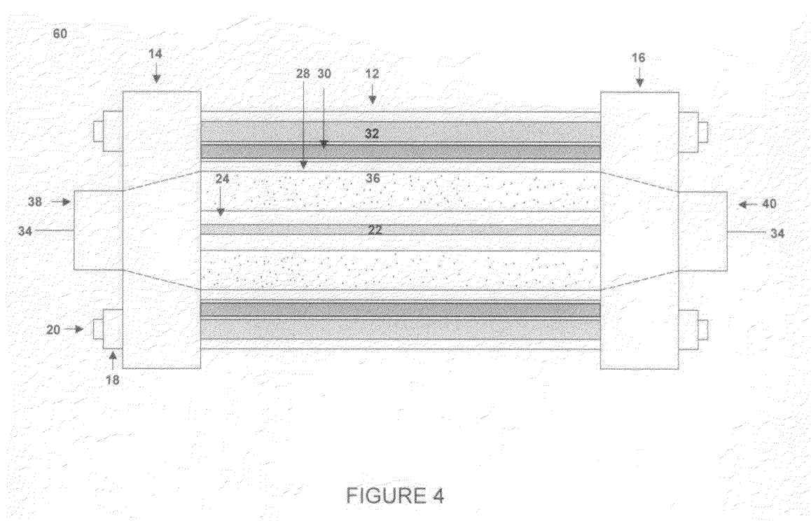

FIG. 4 shows another embodiment of the system for producing a fuel gas from a carbon-containing material, generally designated system 60. As shown in FIG. 4, system 60 is similar in every aspect to the system shown in FIGS. 1 and 2 except that carbon-containing material 26 is not disposed upon dielectric tube 24. Instead, carbon-containing material 26 is entrained in the process stream. Thus, the process stream further comprises carbon-containing material 26. It is understood that system 60 can operate as a continuous system (e.g., circulating or single-pass) with respect to the process stream. If system 60 is a continuous system with a circulating process stream, then system 60 may further comprise a carbon-containing material reservoir through which a circulating process stream may pass to recharge the process stream with carbon-containing material. It is also understood that system 60 can operate with an alternative dielectric-barrier discharge arrangement wherein only one electrode (i.e., either HV electrode 22 or grounded electrode 30) is covered with a dielectric material (i.e., dielectric tube 24 or dielectric tube 28, respectively).

Although FIGS. 1 through 4 are generally cylindrical, the system can be constructed in many other geometries including rectangular. Moreover, one skilled in the art recognizes that the flow rate of the process stream affects the velocity and residence time of the process stream in the system. One skilled in the art recognizes that increasing the process stream's flow rate reduces the fractional production of fuel gas. Moreover, one skilled in the art recognizes that maintaining the same ratio of electrical power to process stream flow rate will maintain the same fractional production of fuel gas. This parameter is especially important to system 60 shown in FIG. 4. One skilled in the art recognizes that increasing the flow rate of the process stream reduces the residence time of carbon-containing material 26 and, thus, reduces the fractional conversion of carbon-containing material 26. Achieving a meaningful production of the fuel gas in a single pass requires a residence time greater than about 1 second.

The system includes heat exchanger 32 to establish and maintain a constant system temperature. One skilled in the art recognizes that increasing the temperature will typically increase the reaction rate of the system and, thus, increase the production of the fuel gas. One skilled in the art also recognizes that the upper system temperature limit will vary with the composition of the process stream. For example, if the system is operating at 1 atmosphere and the process stream comprises hydrogen, then an upper system temperature limit of around 600° C. exists because the fuel gas (i.e., methane) becomes unstable over about 550° C. One skilled in the art also recognizes that the upper system temperature limit will also vary with the system operating pressure. For example, for methane production, increasing the system pressure will increase the high temperature stability limit of methane, the reaction rate of the system (typically), and the production of the fuel gas. The system can operate at a pressure ranging from about a millitorr to about a few atmospheres. However, high pressures present an increased initial cost, maintenance issues, and practical engineering issues.

An advantage of the present invention is its lower operating conditions. For example, the conventional operating conditions required to overcome the activation energy barrier (e.g., about 50-80 kilocalories (“kcal”)/mole) for hydrogasification of coal (C+2H₂→CH₄) (i.e., the process stream comprises hydrogen and the fuel gas comprises methane) are a temperature around 1000° C. and a pressure around 60 atmospheres or higher. The present invention, however, uses the non-thermal plasma to produce hydrogen atoms, which reduce the activation energy barrier. Because the activation energy barrier is lower, the temperature and pressure necessary to overcome the activation energy barrier are also lower.

By considering the above parameters, the present invention also includes a process for producing a fuel gas from a carbon-containing material comprising generating a non-thermal plasma, and contacting the carbon-containing material with the non-thermal plasma for a time and temperature sufficient to form the fuel gas.

The present invention is more particularly described in the following example that is intended as illustrative only because numerous modifications and variations will be apparent to those skilled in the art.

### EXAMPLE 1

EXAMPLE 1 is an embodiment of the invention that was similar to the system shown in FIGS. 1 and 2. The system comprised a coaxial-cylinder dielectric barrier discharge reactor and associated carbon gasifier. The high voltage electrode was a 52 centimeter (“cm”) long stainless steel wire that was inserted into a 50 cm long alumina (e.g., Al₂O₃) ceramic tube with a 0.5 cm outside diameter (“OD”). The grounded electrode was a 30 cm long copper mesh electrode that was placed outside a 45 cm long quartz tube with a 1.68 cm OD. A high voltage alternating current transformer (Eurocom Model 92-0152-70) operating at a frequency of about 450 Hz powered the high voltage stainless steel electrode. The carbon-containing material was an activated carbon powder with a 250 μm mean diameter. One hundred milligrams of the carbon-containing material were placed on the surface of the ceramic tube. The single-pass process stream was 99.99% by volume hydrogen gas flowing at 0.5 liters/minute (“L/min”). Thus, the system was a batch system with respect to the carbon-containing material and a continuous, single-pass system with respect to the process stream. The dielectric barrier discharge gasifier was placed into an electrical oven. Gases produced in the gasification process were collected and analyzed by gas chromatography (GC, Varian CP 3800).

With this embodiment, several samples were taken at various temperatures and hydrogen plasma powers; however all samples were taken at a pressure of 1 standard atmosphere. To test hydrogen plasma powers, the system was stabilized at a temperature of about 25° C. After the temperature was stabilized, the process stream flowed into the system at 0.5 L/min, but non-thermal plasma was not generated because no power was supplied to the HV electrode (i.e., stainless steel wire). The process stream residence time was approximately 8 seconds. After running for 5 minutes, the product stream was collected using a Tedlar bag and analyzed by gas chromatography. After 5 more minutes, 10 watts (“W”) of plasma power was applied to the system by applying a 10 kilovolt (“kV”) voltage. The energy density of the hydrogen plasma was 1200 joules/liter. After 5 minutes, the product stream was collected using a Tedlar bag and analyzed by gas chromatography. This process was repeated at system temperatures including 200, 300, 350, 400, 500, and 575° C.

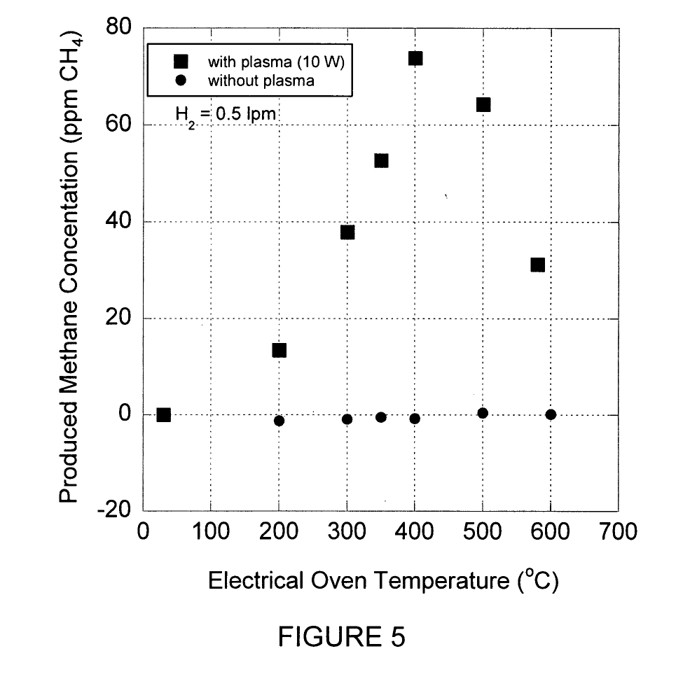

FIG. 5 shows the amount of methane produced from the activated carbon powder by hydrogen gas (closed circles) and by hydrogen plasma (closed squares) at 1 atmosphere. The hydrogen gas (no plasma) produced no methane at all temperatures; however, the 10 W hydrogen plasma produced varying concentrations of methane. The methane concentrations under 10 W hydrogen plasma increased up to 75 parts per million (“ppm”) as the system temperature increased to 400° C. After 500° C., methane production decreased. Although the inventors do not want to be bound by any theory, one possible explanation for the decrease in methane production is that methane is unstable over 550° C. and the thermal decomposition of methane may have been involved at temperatures from 500-600° C. Thus, the system with only hydrogen gas and no plasma produced no methane; conversely, the system with hydrogen gas and plasma produced varying amounts of methane.

Accordingly, it can be seen that this invention provides a system and process for producing a fuel gas from a carbon-containing material. It can be understood that various “active” and “inactive” regions can be established within the reactor using segmented electrodes. It is also understood that the system and process can be used over a wide range of process stream pressures (e.g., a millitorr to a few atmospheres).

Although the description above contains many details, these should not be construed as limiting the scope of the invention but as merely providing illustrations of some embodiments. Therefore, it will be appreciated that the scope of the present invention fully encompasses other embodiments which may become obvious to those skilled in the art, and that the scope of the present invention is accordingly to be limited by nothing other than the appended claims. All structural, chemical, and functional equivalents to the elements of the above-described embodiments that are known to those of ordinary skill in the art are expressly incorporated herein by reference and are intended to encompass the present claims. Moreover, it is not necessary for a device, system, method, or process to address each and every problem sought to be solved by the present invention for it to be encompassed by the present claims.

We claim:

1 .A system for producing a fuel gas from a carbon-containing material comprising
a non-thermal plasma generator;
an electric power source for providing an electrical charge to said non-thermal plasma generator;
a process stream inlet configured to inject a process stream into said non-thermal plasma generator; and
a product stream outlet configured to remove a product stream from said non-thermal plasma generator
wherein said process stream comprises hydrogen, steam, or carbon dioxide, and said product stream comprises said fuel gas.

2. The system of claim 1 wherein said non-thermal plasma generator comprises
a high voltage electrode;
a grounded electrode spaced apart from said high voltage electrode;
a first dielectric layer between said high voltage electrode and said grounded electrode; and
a modification passage between said high voltage electrode and said grounded electrode
wherein said process stream inlet is configured to inject said process stream into said modification passage, and said high voltage electrode is connected to said electric power source and is characterized as energizable to create non-thermal electrical microdischarges between said high voltage electrode and said grounded electrode across said first dielectric layer.

3. The system of claim 2 wherein said carbon-containing material is within said modification passage.

4. The system of claim 3 wherein said fuel stream comprises methane.

5. The system of claim 3 wherein said first dielectric layer is adjacent to said high voltage electrode and said modification passage is between said first dielectric layer and said grounded electrode.

6. The system of claim 5 wherein said high voltage electrode, said first dielectric layer, said modification passage, and said grounded electrode are concentric to each other.

7. The system of claim 3 wherein said first dielectric layer is adjacent to said grounded electrode and said modification passage is between said first dielectric layer and said high voltage electrode.

8. The system of claim 7 wherein high voltage electrode, gas modification passage, said first dielectric layer, and said grounded electrode are concentric to each other.

9. The system of claim 3 further comprising a second dielectric layer wherein said first dielectric layer is adjacent to said high voltage electrode, said second dielectric layer is adjacent to said grounded electrode, and said modification passage is between said first dielectric layer and said second dielectric layer.

10. The system of claim 9 wherein said high voltage electrode, said first dielectric layer, said modification passage, said second dielectric layer, and said grounded electrode are concentric to each other.

11. The system of claim 2 wherein said process stream further comprises said carbon-containing material.

12. The system of claim 11 wherein said fuel stream comprises methane.

13. The system of claim 11 wherein said first dielectric layer is adjacent to said high voltage electrode and said modification passage is between said first dielectric layer and said grounded electrode.

14. The system of claim 13 wherein said high voltage electrode, said first dielectric layer, said modification passage, and said grounded electrode are concentric to each other.

15. The system of claim 11 wherein said first dielectric layer is adjacent to said grounded electrode and said modification passage is between said first dielectric layer and said high voltage electrode.

16. The system of claim 15 wherein said high voltage electrode, said modification passage, said first dielectric layer, and said grounded electrode are concentric to each other.

17. The system of claim 11 further comprising a second dielectric layer wherein said first dielectric layer is adjacent to said high voltage electrode, said second dielectric layer is adjacent to said grounded electrode, and said modification passage is between said first dielectric layer and said second dielectric layer.

18. The system of claim 17 wherein said high voltage electrode, said first dielectric layer, said modification passage, said second dielectric layer, and said grounded electrode are concentric to each other.

19. A process for producing a fuel gas from a carbon-containing material comprising
generating a non-thermal plasma; and
contacting said carbon-containing material with said non-thermal plasma at a pressure and a temperature sufficient to form a product stream comprising said fuel gas.

20. The process of claim 19 wherein said fuel gas comprises methane and said temperature is about 200-600° C.
